(19) United States
(12) Patent Application Publication
(10) Pub. No.: US 2007/0270443 A1
(43) Pub. Date: Nov. 22, 2007

(54) Methods and compositions for the treatment of viral infections

(76) Inventors: Gregory Went, Mill Valley, CA (US); David Chernoff, San Anselmo, CA (US); Paul Spence, Mill Valley, CA (US); Terry Burkoth, San Francisco, CA (US); Timothy Fultz, Pleasant Hill, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/804,753

(22) Filed: May 18, 2007

Related U.S. Application Data
(60) Provisional application No. 60/801,900, filed on May 19, 2006.
(60) Provisional application No. 60/835,621, filed on Aug. 4, 2006.

(51) Int. Cl. A61K 31/522; A61K 31/13

(52) U.S. Cl. 514/263.32; 514/661

(57) ABSTRACT

Compositions for treating flu comprise an M2 inhibitor, and optionally a neuraminidase inhibitor, wherein at least one of said M2 inhibitor or said neuraminidase inhibitor is provided in an extended release dosage form.

### CROSS-REFERENCE TO RELATED APPLICATIONS

Priority is claimed to U.S. Provisional Application Nos. 60/801,900, filed May 19, 2006, and 60/835,621, filed Aug. 4, 2006; both applications are incorporated herein by reference in their entireties.

### BACKGROUND OF THE INVENTION

This invention relates to methods and compositions for treating viral infections, and has particular reference to the treatment of flu.

Influenza, more commonly known as the flu, is an acute, viral infection that attacks mainly the upper respiratory tract—the nose, throat and bronchi and rarely also the lungs. Although the flu is considered to be an infection of the respiratory tract, individuals suffering from the flu usually become acutely ill with high fever, chills, headache, weakness, loss of appetite and aching joints. The typical length of time from when a person is exposed to influenza virus to when symptoms first occur ranges between one and five days, with an average of two days. Adults can be infectious (i.e., shedding virus) starting the day before the onset of symptoms begin until approximately 5 days after the onset of illness. Children can be infectious for longer periods of time. Systemic symptoms include abrupt onset of fever (e.g. usually 100-103 degrees F. in an adult and possibly higher in children), chills, headaches, myalgia and malaise.

Most people recover within one to two weeks without requiring any medical treatment. However, in the very young, the elderly and people suffering from medical conditions such as lung diseases, diabetes, cancer, kidney or heart problems, influenza poses a serious risk. In these people, the infection may lead to severe complications of underlying diseases, pneumonia and death.

Also, influenza infections are known to increase the susceptibility of an infected to particular bacterial infections caused by species of bacterial pathogens such as, the pneumococcus, staphylococcus, mycoplasma, non-group H. influenza, and Moraxella catarrhalis. Secondary bacterial infections, such as, but not limited to infections of the lower respiratory tract (e.g., pneumonia), middle ear infections (e.g., otitis media) and bacterial sinusitis are common complications of an infection with viral influenza.

Given that the flu and its associated complications (e.g. bacterial infections, viral pneumonia, and cardiac and other organ system abnormalities) represent the sixth leading cause of death in the world and the leading infectious cause of death, there is a clear need for improved therapeutics and methods for the treatment of viral diseases and disorders, such as the flu and its related conditions.

### SUMMARY OF THE INVENTION

The present invention provides methods and compositions for treating and preventing viral infections, particularly influenza, using an M2 channel inhibitor optionally in combination with a neuraminidase inhibitor. Such compositions and methods may be used to reduce the course and severity of the disease, and decrease the emergence of drug resistant strains. The compositions of the invention may be used for treating human or non-human animal patients infected with Influenza A, including highly virulent strains of Influenza A such, for example, as H5N1.

The present invention also comprehends the use of an M2 channel inhibitor and optionally a neuraminidase inhibitor in the manufacture of a medicament for use in the treatment or prophylaxis of a viral infection, in particular influenza, e.g., Influenza A.

Optionally, the M2 inhibitor, the neuraminidase inhibitor, or both are present at a higher dose than that typically administered to a subject for treatment of influenza. For example, the amount of amantadine (an M2 inhibitor) may be 200-400 mg per day rather than the typical 100-200 mg per day (i.e. without the improved formulations described herein). Alternatively, when used in combination, lower or reduced amounts of the M2 inhibitor and/or the neuraminidase inhibitor are in a unit dose relative to the amount of each agent when administered as a monotherapy. In a preferred embodiment, the amount of the M2 inhibitor in the pharmaceutical composition is equal to or greater than the amount typically administered as a monotherapy and the amount of the neuraminidase inhibitor is less than the amount typically administered.

Although use of an M2 inhibitor together with a second antiviral agent, such as a neuraminidase inhibitor, has been disclosed (e.g. Govorkova et. al., Antimicrobial Agents and Chemother. 48(12):4855-63 (2004)), the problem of providing release of the M2 inhibitor in a desired manner (e.g. in an amount high enough to treat symptoms or damaging effects of an underlying disease while avoiding undesirable side effects e.g. CNS side effects) when present as a combined therapy has not been addressed. In particular, the presently available dosage forms of M2 inhibitors need to be administered frequently and require dose escalation at the initiation of therapy to avoid side effects associated with initial exposure to the therapeutic agent. M2 inhibitors such as amantadine must be dosed twice daily (BID) owing to their CNS side effects, despite having relatively long half lives. This leads to difficulty in achieving adequate patient compliance. This problem has not been addressed in the context of providing an M2 inhibitor as a combined therapy.

In one embodiment of the invention, the M2 inhibitor, the neuraminidase inhibitor, or both agents may be provided in an extended release form with or without an immediate release component in order to maximize the therapeutic benefit of each, while reducing unwanted side effects associated with each. Advantageously, the M2 inhibitor, and optionally the neuraminidase inhibitor, is formulated with a polymer matrix and/or polymer coating that controls release of the active agent(s) and achieves a desired release profile.

As used herein, “immediate release formulation” refers to a formulation of an active pharmaceutical ingredient that releases greater than 80 percent of the active pharmaceutical ingredient in less than one hour in the USP dissolution method. Typically, the release of the active ingredient in an immediate release (IR) formulation is greater than 80 percent in less than 30 minutes as in FIG. 1. The term “extended release dosage form” (also referred to in the art as “controlled release” and “sustained release” dosage forms) refers to dosage forms where the active drug substance or substances are released over an extended period of time. An extended release dosage form may or may not comprise an immediate release component.

Optionally, the composition described herein is formulated such that at least one of said M2 inhibitor or said neuraminidase inhibitor has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium.

As used herein, the term “Cmax” refers to the maximum concentration reached by a given dose of drug in a biological sample (e.g. blood, serum, and cerebrospinal fluid). The term “Cmean” refers to the average concentration of the drug in the sample over time. Cmax and Cmean may be further defined to refer to specific time periods relative to administration of the drug. The time required to reach the maximal concentration (“Cmax”) in a particular patient sample type is referred to as the “Tmax.” The agents of the combination are administered in formulations that reduce the variability of the ratio of the concentrations of the active agents over a period of time, thereby optimizing the antiviral effect of the agents and maximizing their therapeutic benefit while minimizing the side effects.

In a preferred embodiment, the dosage form is provided in a non-dose escalating form, preferably in a twice per day or once per day form. In such cases, the concentration ramp (or Tmax effect) may be reduced so that the change in concentration as a function of time (dC/dT) is altered to reduce or eliminate the need to dose escalate the drug. A reduction in dC/dT may be accomplished, for example, by increasing the Tmax in a relatively proportional manner. For example, a two-fold increase in the Tmax value may reduce dC/dT by approximately a factor of 2.

The provision of such non-dose escalating dosage forms are particularly useful as they provide the drug at a therapeutically effective amount from the onset of therapy further improving patient compliance and adherence and enable the achievement of a therapeutically effective steady-state concentration of the drug in a shorter period of time. This results in an earlier indication of effectiveness and increasing the utility of these therapeutic agents for diseases and conditions where time is of the essence. Furthermore, the compositions of the present invention, by virtue of their design, allow for higher doses of the drug to be safely administered, again increasing the utility of these agents for a variety of viral indications, reducing the probability of disease resistance strains, and dramatically improving our ability to effectively manage flu and flu pandemics.

If desired, the M2 inhibitor or the neuraminidase inhibitor of the combination is released into a subject sample at a slower rate than observed for an IR formulation of the same quantity of the antagonist. The release rate is measured as the dC/dT over a defined period within the period of 0 to Tmax for the IR formulation and the dC/dT rate is less than about 80% of the rate for the IR formulation. In some embodiments, the dC/dT rate is less than about 60%, 50%, 40%, 30%, 20%.or 10% of the rate for the IR formulation. Similarly, the neuraminidase inhibitor may also be released into a patient sample at a slower rate than observed for an IR formulation of the same quantity wherein the release rate is measured as the dC/dT over a defined period within the period of 0 to Tmax for the IR formulation and the dC/dT rate is less than about 80%, 60%, 50%, 40%, 30%, 20%, or 10%, of the rate for the IR formulation of the same M2 inhibitor over the first 1, 2, 4, 6, 8, 10, or 12 hours.

Optionally, the extended release formulations exhibit plasma concentration curves having initial (e.g., from 2 hours after administration to 4 hours after administration) slopes less than 75%, 50%, 40%, 30%, 20% or 10% of those for an IR formulation of the same dosage of the same M2 inhibitor.

The ratio of the concentrations of two agents in a combination is referred to as the “Cratio,” which may fluctuate as the combination of drugs is released, transported into the circulatory system or CNS, metabolized, and eliminated. An objective of the present invention is to stabilize the Cratio for the combinations described herein. In some embodiments, it is preferred to reduce or even minimize the variation in the Cratio (termed “Cratio,var”). Employing the methods described herein, the release profiles of each active pharmaceutical ingredient may be modified to produce nearly constant Cratios, thereby minimizing Cratio, var. In cases where the Tmax and T1/2 of the M2 inhibitor and the neuraminidase inhibitor are markedly different, e.g. by a factor of two or more, the desired release profiles will likely be dissimilar in order to minimize the relative variability of the active agents between doses.

The present invention therefore features formulations of combinations directed to dose optimization or release modification to reduce adverse effects associated with separate administration of each agent. The combination of the M2 inhibitor and the neuraminidase inhibitor may result in an additive or synergistic response, as described below.

In all foregoing aspects of the invention, at least 50%, 80, 90%, 95%, or essentially all of the M2 inhibitor in the pharmaceutical composition may be provided in an extended release dosage form. In some embodiments, at least 99% of the M2 inhibitor remains in the extended dosage form one hour following introduction of the pharmaceutical composition into a subject. The M2 inhibitor may have a Cₘₐₓ/Cₘₑₐₙ of approximately 2, 1.6, 1.5, 1.4, 1.3, 1.2 or less, approximately 2 hours to at least 8, 12, 16, 24 hours after the M2 inhibitor is introduced into a subject. The neuraminidase inhibitor may also be provided in an extended release dosage form. Thus, at least 50%, 60%, 70%, 80%, 90%, 95%, or essentially all of the neuraminidase inhibitor may be provided as an extended release formulation. If provided as such, the neuraminidase inhibitor may have a Cₘₐₓ/Cₘₑₐₙ of approximately 2, 1.6, 1.5, 1.4, 1.3, 1.2 or less, approximately 2 hours to at least 6, 8, 12, 16, or 24 hours after the neuraminidase inhibitor is introduced into a subject.

Optionally, the Cratio,var of the M2 inhibitor and the neuraminidase inhibitor is less than 100%, e.g., less than 70%, 50%, 30%, 20%, or 10% after the agents have reached steady-state conditions. Optionally, the Cratio,var of the M2 inhibitor and the neuraminidase inhibitor is less than 100%, e.g. less than 70%, 50%, 30%, 20%, or 10% during the first 24 hours post-administration of the agents. In some embodiments, the Cratio,var is less than about 90% (e.g., less than about 75% or 50%) of that for IR administration of the same active pharmaceutical ingredients over the first 4, 6, 8, or 12 hours after administration.

In all foregoing aspects of the invention, the M2 inhibitor may be an aminoadamantane derivative, for example rimantadine or amantadine, and pharmaceutically acceptable salts thereof. The neuraminidase inhibitor may be oseltamivir, zanamivir, or peramivir, and pharmaceutically acceptable salts thereof. In a particular embodiment, the M2 inhibitor is amantadine and the neuraminidase inhibitor is oseltamivir.

In some embodiments, the M2 inhibitor, the neuraminidase inhibitor, or both agents are formulated for oral, enteral, intravenous, topical, intranasal, subtopical transepithelial, subdermal, or inhalation delivery. Thus, the agents described herein may be formulated as a suspension, capsule, tablet, suppository, lotion, patch, or device (e.g., a subdermally implantable delivery device or an inhalation pump). If desired, the M2 inhibitor and the neuraminidase inhibitor may be admixed in a single composition. Alternatively, the two agents are delivered in separate formulations sequentially, or within one hour, two hours, three hours, six hours, 12 hours, or 24 hours of each other. Optionally, the two agents are provided together in the form of a kit. Preferably, the M2 inhibitor and the neuraminidase inhibitor are provided in a unit dosage form.

The invention also comprehends a method of preventing or treating flu or a flu-related condition comprising administering to a subject in need thereof a therapeutically effective amount of a pharmaceutical comprising amantadine or rimantadine or a pharmaceutically acceptable salt thereof in an extended release dosage form, wherein the amantadine or rimantadine has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium.

Further, the invention comprehends the use of a composition comprising amantadine or rimantadine or a pharmaceutically acceptable salt thereof in an extended release dosage form in the manufacture of a medicament for preventing or treating flu or a flu-related condition in a subject in need thereof, wherein the amantadine or rimantadine has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium.

In various embodiments the amantadine or rimantadine or pharmaceutically acceptable salt thereof has a Cₘₐₓ/Cₘₑₐₙ of approximately 1.6 or less approximately 2 hours to at least 12 hours after said composition is introduced into a human.

Upon oral administration to a human, the mean plasma concentration profile of the amantadine, rimantadine, or pharmaceutically acceptable salt thereof, may have a slope from 2 hours to 4 hours after administration that is less than 50% of that for an IR formulation of the amantadine, rimantadine, or the same pharmaceutically acceptable salt thereof at the same administered dose.

In various embodiments, a human subject may be administered the amantadine or rimantadine or pharmaceutically acceptable salt thereof in an amount of 50-1000 mg, 150-900 mg, 250-850 mg, 300-800 mg, 350-750 mg, 400-750 mg, 450-700 mg, 500-650 mg, or 550 to 600 mg per day, or in amounts of at least about 200 mg, 300 mg, 400 mg, 500 mg, 600 mg or 700 mg per day.

### BRIEF DESCRIPTION OF THE FIGURES

FIG. 1 is a graph showing dissolution profiles for immediate and extended release formulations of amantadine. The extended release formulations (designated Amantadine SR) contain 200-220 mg of amantadine. These dissolution profiles can be obtained from a USP II Paddle system using water as the medium.

FIG. 2 is a graph showing predicted plasma blood levels for 24 hours of dosing with an IR formulation of amantadine dosed b.i.d. and extended release formulations of amantadine dosed q.d., obtained using the Gastro-Plus software package v.4.0.2. The extended release formulations contain 200-220 mg of amantadine as indicated.

### DETAILED DESCRIPTION OF SPECIFIC EMBODIMENTS OF THE INVENTION

The present invention provides methods and compositions for treating or preventing flu and flu-related conditions (e.g. a bacterial infection associated with the flu). The flu or flu-related condition may be caused by infection with influenza A, B or C. In a particular embodiment, the infection is caused by influenza A.

The combination of the present invention includes a first agent that is an M2 inhibitor and a second agent that is a neuraminidase inhibitor (e.g., oseltamivir/TAMIFLU®, zanamivir/RELENZA®). Optionally, the combination may comprise more than one M2 inhibitor and/or more than one neuraminidase inhibitor. Further active ingredients (e.g. antibiotics, analgesics, decongestants etc.) may also be employed in conjunction with the combination.

The combination is administered such that the symptoms associated with flu or a flu-related condition are alleviated or prevented, or alternatively, such that progression of the flu or flu-related condition is reduced. Desirably, one or both agents is formulated for extended release. Preferably, the compositions of the present invention are formulated to provide a concentration ratio variability over the dosing interval that is less than that observed or predicted for formulations where neither component or only one component is in an extended release form.

A preferred M2 inhibitor of the invention is amantadine, which is described, for example, in U.S. Pat. Nos. 3,152,180; 5,891,885; 5,919,826; and 6,187,338. Additional aminoadamantane compounds that can be used in the invention are described, for example, in U.S. Pat. Nos. 4,346,112; 5,061,703; 5,334,618; 5,382,601; 6,444,702; 6,620,845; and 6,662,845, incorporated herein by reference.

The pharmaceutical composition may be formulated to provide amantadine in an amount ranging between 25 and 800 mg/day, 100 and 600 mg/day, 200 and 500 mg/day, 200 and 400 mg/day, or 300 and 400 mg/day; or rimantadine in an amount ranging between 50 and 400 mg/day, 75 and 300 mg/day, 150 and 300 mg/day, 100 and 250 mg/day, or 100 and 200 mg/day.

Table 1 shows exemplary the pharmacokinetic properties (e.g., Tmax and T1/2) of amantadine and rimantadine.

TABLE 1
Pharmacokinetics and Toxicity in humans for selected M2 inhibitors

| | Human PK | Tmax | | Dose Dependent |
|---|---|---|---|---|
| Compound | (t½) in hrs | in hrs | Normal Dose | Toxicity |
| Amantadine | 15 | 3 | 100-300 mg/day | Hallucination |
| Rimantadine | 25 | 6 | 100-200 mg/day | Insomnia |

Neuraminidase inhibitors that can be used in the invention include oseltamivir phosphate (GS4104, Tamiflu®; Roche/Gilead), zanamivir (GG167, RELENZA® &; GlaxoSmithKline) and RWJ-270201 (BCX-1812, BioCryst). Oseltamivir, described in U.S. Pat. No. 5,763,483, is presently approved by the United States FDA for the treatment of flu.

The pharmaceutical composition may be formulated to provide oseltamivir in an amount ranging between 25 and 250 mg/day, 35 and 210 mg/day, 50 and 200 mg/day, 60 and 180 mg/day, 75 and 150 mg/day, or 80 and 120 mg/day; or zanamivir in an amount ranging between 2 and 40 mg/day, 4 and 30 mg/day, 8 and 25 mg/day, or 10 and 20 mg/day.

Table 2 shows exemplary the pharmacokinetic properties (e.g., Tmax and T1/2) of oseltamivir and zanamivir.

TABLE 2
Pharmacokinetics and Tox in humans for selected
Neuraminidase inhibitors

| | Human | | | Main Dose- |
|---|---|---|---|---|
| | PK T½ | Tmax | Normal | Dependent |
| Compound | (hrs) | (hrs) | Dose | Adverse Event |
| oseltamivir/TAMIFLU ® | 3 | 3-4 | 75-100 | Nausea, vomiting, |
| | | | mg/day | diarrhea, headache |
| zanamivir/RELENZA ® | 2.5-5.1 | 1-2 | 10-20 | Diarrhea, nausea, |
| | | | mg/day | vomiting, sinusitis |

A pharmaceutical composition according to the invention is prepared by combining a desired active agent, i.e. an M2 inhibitor and/or neuraminidase inhibitor, with one or more additional ingredients that, when administered to a subject, causes the active agent to be released at a targeted concentration range for a specified period of time. For example, the M2 inhibitor and/or neuraminidase inhibitor may be formulated with a polymer matrix and/or polymer coating that controls release of the active agent(s) and achieves a desired release profile. The active agent is formulated so that it is released at a dC/dT that is significantly reduced over the IR dosage form, with an associated delay in the Tmax. For amantadine and rimantadine, the compared to IR forms are SYMMETREL® (amantadine hydrochloride, USP) and FLUMADINE® (rimantadine hydrochloride) tablets, respectively. For oseltamivir, the compared to IR dosage form is TAMIFLU® (oseltamivir phosphate) capsules.

In certain embodiments, a reduction in dC/dT is accomplished by releasing less than 30%, 50%, 75%, 90%, or 95% of the M2 inhibitor and/or neuraminidase inhibitor into the circulatory or neural system within one hour of such administration. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. and/or neuraminidase inhibitor. In addition, the M2 inhibitor and/or neuraminidase inhibitor may be provided such that it is released at a rate resulting in a Cₘₐₓ/Cₘₑₐₙ of approximately 2 or less for approximately 2 hours to at least 8 hours after the agent is introduced into a subject. Optionally, the extended release formulations exhibit plasma concentration curves having initial (e.g., from 2 hours after administration to 4 hours after administration) slopes less than 75%, 50%, 40%, 30%, 20% or 10% of those for an IR formulation of the same dosage of the same M2 inhibitor or neuraminidase inhibitor. The determination of initial slopes of plasma concentration is described in U.S Pat. No. 6,913,768, hereby incorporated by reference.

Optionally, the composition described herein is formulated such the M2 inhibitor has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium. The USP dissolution method is described in the U.S. Pharmacopeia and National Formulary (USP29-NF24, Rockville, Md. U.S. Pharmacopeial Convention, Inc.; 2006).

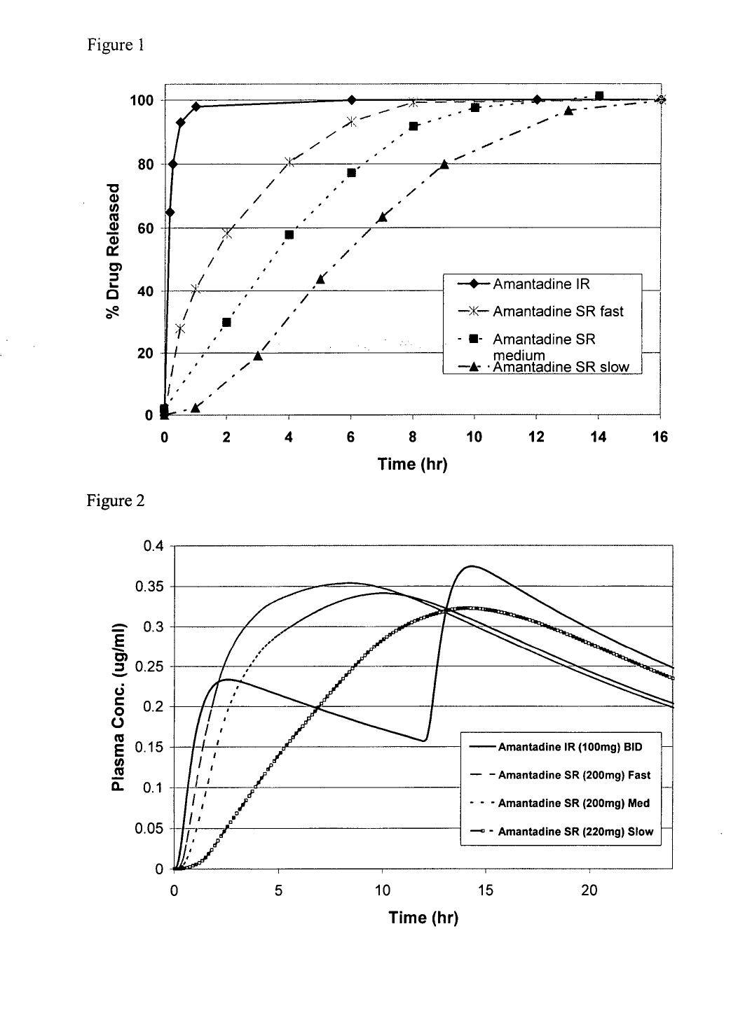

Desirably, the compositions described herein have an in vitro dissolution profile that is substantially identical to the dissolution profile shown for the extended release formulations shown in FIG. 1, upon administration to a subject at a substantially constant daily dose, achieves a plasma concentration profile that is substantially identical to those shown in FIG. 2.

A release profile, i.e., the extent of release of the M2 inhibitor or the neuraminidase inhibitor over a desired time, can be determined for a given time by calculating the Cₘₐₓ/Cₘₑₐₙ for a desired time range. For example, the M2 inhibitor and/or the neuraminidase inhibitor can be provided so that it is released at Cₘₐₓ/Cₘₑₐₙ of approximately 2 or less for approximately 2 hours to at least 6 hours after introduction into a subject. In various embodiments, the M2 inhibitor and/or the neuraminidase inhibitor may be formulated as an extended release composition having a Cmax /Cmean of approximately 2.5, 2, 1.5, or 1.0 approximately 1, 1.5, 2 hours to at least 6, 8, 9, 12, 18, 21, or 24 hours after the introduction.

Using the formulations described herein, therapeutic levels may be achieved while minimizing debilitating side-effects that are usually associated with immediate release formulations. Furthermore, as a result of the reduction in the time to obtain peak plasma level and the potentially extended period of time at the therapeutically effective plasma level, the dosage frequency may be reduced to, for example, once or twice daily dosage, thereby improving patient compliance and adherence. For example, side effects including psychosis and cognitive deficits associated with the administration of M2 inhibitors may be lessened in severity and frequency through the use of extended release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing. With regards to the neuraminidase inhibitor, the lower dC/dT and Cmean will result in a lower incidence of cardiovascular or gastric side effects and other adverse events.

The amounts and ratios of the M2 inhibitor and the neuraminidase inhibitor are conveniently varied to maximize the therapeutic benefit and minimize the toxic or safety concerns. In one example, the amount of amantadine ranges between 100 and 200 mg per day and the amount of oseltamivir ranges between 80 and 120 mg/day.

When the amantadine is in an extended release form, the preferred dosage range is 200 mg to 400 mg per day; daily doses of about 200, 220, 240, 260, 280, 300, 320, 340, 360, 380 and 400 mg are particularly preferred. When the oseltamivir is in an extended release form, the preferred dosage range 75 mg to 150 mg per day; daily doses of about 80, 85, 90, 95, 100, 105, 110, 120, 130, 140, or 150 mg per day are particularly preferred. In a particularly preferred embodiment the amantadine dose is 200-400 mg per day, taken in combination with an oseltamivir dose of 75-150 mg/day, administered as a single dosage form, with no dose escalation over time. The combination dosage form preferably has extended release formulations for amantadine, oseltamivir or both, such that the dissolution profile of the two drugs in the combination are “matched”, especially with regards to the Tmax, dC/dT (normalized for the dose of M2 inhibitor and neuraminidase inhibitor) in a human. For amantadine and oseltamivir, which have substantially different pharmacokinetic properties, in vitro dissolution profiles may be different to optimize this matching.

In a preferred embodiment of the invention, amantadine and oseltamivir are formulated into beads or pellets, described herein, with dissolution profiles providing extended release of each drug. More preferably, beads or pellets of amantadine are prepared with dissolution profiles similar to that shown for amantadine in FIG. 1 and, separately, beads or pellets of oseltamivir are prepared with a dissolution profile that is somewhat slower than that for the amantadine. The preferred pellets are approximately 0.4 mg each and contain approximately 60 μg amantadine or oseltamivir and easily characterized by known methods. The beads may be filled into gelatin capsules by mass or number to achieve the preferred mass of amantadine of 100-200 mg per capsules and oseltamivir of 75-150 mg per capsule. For example, a 600 mg amantadine 45 mg oseltamivir combination may be prepared by combining 1000 amantadine beads with 750 oseltamivir beads in each capsule, equivalent to 400 mg amantadine beads plus 300 mg oseltamivir beads per capsule.

Additionally, different release profiles for each active pharmaceutical ingredient may be prepared and combined in prescribed ratios to adjust the release profile for each of the ingredients, enabling the more rapid development of formulations for development purposes or specialized formulations for individual products.

In certain embodiments, the combinations provide additive effects. Additivity is achieved by combining the active agents without requiring extended release technologies. In other embodiments, particularly when the pharmacokinetic profiles of the combined active pharmaceutical ingredients are dissimilar, extended release formulations optimize the pharmacokinetics of the active pharmaceutical agents to reduce the variability of the Cratio over time. Reduction of Cratio variability over a defined time period enables a concerted effect for the agents over that time, maximizing the effectiveness of the combination. The Cratio variability (“Cratio.var”) is defined as the standard deviation of a series of Cratios taken over a given period of time divided by the mean of those Cratios multiplied by 100%. The Cratio for the extended release formulation of drugs with significantly different pharmacokinetic properties is more consistent than for the IR administration of the same drugs over any significant time period, including shortly after administration and at steady state.

The combination of the invention may be administered in either a local or systemic manner or in a depot or extended release fashion. The agents may be delivered in an oral, transdermal or intranasal formulation. The same or different administration routes may be used for the M2 inhibitor and the neuraminidase inhibitor. In a preferred embodiment, the M2 inhibitor, the neuraminidase inhibitor of the combination, or both agents, may be formulated to provide extended release. For example, a pharmaceutical composition that provides extended release of the M2 inhibitor, the neuraminidase inhibitor, or both may be prepared by combining the desired agent or agents with one or more additional ingredients that, when administered to a subject, causes the respective agent or agents to be released at a targeted rate for a specified period of time. The two agents are preferably administered in a manner that provides the desired effect from the first and second agents in the combination. Optionally, the first and second agents are admixed into a single formulation before they are introduced into a subject. The combination may be sub-divided in unit doses containing appropriate quantities of the first and second agents. The unit dosage form may be, for example, a capsule or tablet itself or it can be an appropriate number of such compositions in package form.

The preparation of pharmaceutical or pharmacological compositions is known to those of skill in the art. General techniques for formulation and administration are found in “Remington: The Science and Practice of Pharmacy, Twentieth Edition,” Lippincott Williams & Wilkins, Philadelphia, Pa., incorporated herein by reference. Tablets, capsules, pills, powders, granules, dragees, gels, slurries, ointments, solutions suppositories, injections, inhalants and aerosols are examples of such formulations.

Combinations can be provided as pharmaceutical compositions that are optimized for particular types of delivery. For example, pharmaceutical compositions for oral delivery are formulated using pharmaceutically acceptable carriers that are well known in the art and described further below. The carriers enable the agents in the combination to be formulated, for example, as a tablet, pill, capsule, solution, suspension, powder, liquid, or gel for oral ingestion by the subject.

The M2 inhibitor, the neuraminidase inhibitor of the invention, or both agents may be provided in an extended release form. In one example, at least 50%, 90%, 95%, 96%, 97%, 98%, 99%, or even in excess of 99% of the M2 inhibitor is provided in an extended release dosage form.

As shown in Tables 1 and 2, the pharmacokinetic half-lives of the drugs of both classes can vary. Thus, suitable formulations may be conveniently selected to achieve nearly constant concentration profiles over an extended period (preferably from 8 to 24 hours) thereby maintaining both agents in a constant ratio and concentration for optimal therapeutic benefits for both acute and chronic administration. Preferred Cratio,var values may be less than about 30%, 50%, 75%, 90% of those for IR administration of the same active pharmaceutical ingredients over the first 4, 6, 8, or 12 hours after administration. Preferred Cratio,var values are less than about 100%, 70%, 50%, 30%, 20%, 10%.

Formulations that deliver this constant, measurable profile also allow one to achieve a monotonic ascent from an acute ratio to a desired chronic ratio for drugs with widely varying elimination half-lives. Compositions of this type and methods of treating patients with these compositions are embodiments of the invention. Numerous ways exist for achieving the desired release profiles, as exemplified below.

Suitable methods for preparing combinations in which the M2 inhibitor and/or neuraminidase inhibitor are provided in extended release-formulations include those described in U.S. Pat. No. 4,606,909 (hereby incorporated by reference). This reference describes an extended release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve prills of pharmaceutically acceptable material (e.g., sugar/starch, salts, and waxes) may be coated with a water permeable polymeric matrix containing an M2 inhibitor and next overcoated with a water-permeable film containing dispersed within it a water soluble particulate pore forming material.

One or both agents of the combination may additionally be prepared as described in U.S. Pat. No. 4,897,268, involving a biocompatible, biodegradable microcapsule delivery system. Thus, the M2 inhibitor may be formulated as a composition containing a blend of free-flowing spherical particles obtained by individually microencapsulating quantities of amantadine, for example, in different copolymer excipients which biodegrade at different rates, therefore releasing amantadine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybuterate and copolymers thereof, polymaleamides, copolyaxalates and polysaccharides.

In one embodiment of the invention, the M2 inhibitor, the neuraminidase inhibitor, or both agents may be provided in an extended release form with or without an immediate release component in order to maximize the therapeutic benefit of each, while reducing unwanted side effects associated with each. When these drugs are provided in an oral form without the benefit of extended or extended release components, they are released and transported into the body fluids over a period of minutes to several hours. Thus, the composition of the invention may contain an M2 inhibitor and an extended release component, such as a coated extended release matrix, an extended release matrix, or an extended release bead matrix. In one example, amantadine (e.g., 100-400 mg) is formulated without an immediate release component using a polymer matrix (e.g., Eudragit), Hydroxypropyl methyl cellulose (HPMC) and a polymer coating (e.g., Eudragit). Such formulations can, for example, be compressed into solid tablets or granules or formed into pellets for capsules or tablets. Optionally, a coating such as Opadry® or Surelease® is used.

Separately prepared pellets, preferably release controlling pellets, combined in any manner provide the flexibility of making ratios of M2 inhibitor to neuraminidase inhibitor containing compositions ranging from 1:1000 to 1000:1, more preferably from 1:100 to 100:1, even more preferably 1:10 to 10:1 most preferably 1:4 to 4:1 by mass or by numbers of pellets (see Example 7), and at the desired release profiles for each of the active ingredients.

Optionally, the M2 inhibitor, the neuraminidase inhibitor, or both agents are prepared using the OROS® technology, described for example, in U.S. Pat. Nos. 6,919,373, 6,923,800, 6,929,803, 6,939,556, and 6,930,128, all of which are hereby incorporated by reference. This technology employs osmosis to provide precise, extended drug delivery for up to 24 hours and can be used with a range of compounds, including poorly soluble or highly soluble drugs. OROS® technology can be used to deliver high drug doses meeting high drug loading requirements. By targeting specific areas of the gastrointestinal tract, OROS® technology may provide more efficient drug absorption and enhanced bioavailability. The osmotic driving force of OROS® and protection of the drug until the time of release eliminate the variability of drug absorption and metabolism often caused by gastric pH and motility.

Alternatively, the combination may be prepared as described in U.S. Pat. No. 5,395,626 features a multilayered extended release pharmaceutical dosage form. The dosage form contains a plurality of coated particles wherein each has multiple layers about a core containing an M2 inhibitor and/or the neuraminidase inhibitor whereby the drug containing core and at least one other layer of drug active is overcoated with an extended release barrier layer therefore providing at least two extended releasing layers of a water soluble drug from the multilayered coated particle.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm dil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticized according to the properties of the coating blend such as the glass transition temperature of the main agent or mixture of agents or the solvent used for applying the coating compositions. Suitable plasticizers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifungal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, may also be used as a carrier.

Additional methods for making extended release formulations are described in, e.g., U.S. Pat. Nos. 5,422,123; 5,601,845; 5,912,013; and 6,194,000, all of which are hereby incorporated by reference.

Alternatively, the compositions of the present invention may be administered transdermally. Preparation for delivery in a transdermal patch can be performed using methods also known in the art, including those described generally in, e.g., U.S. Pat. Nos. 5,186,938; 6,183,770; 4,861,800; 4,284,444 and WO 89/09051. A patch is a particularly useful embodiment in cases where the therapeutic agent has a short half-life or requires reduction in dC/dT. Patches can be made to control the release of skin-permeable active ingredients over a 12 hour, 24 hour, 3 day, and 7 day period. In one example, a 2-fold daily excess of an M2 inhibitor is placed in a non-volatile fluid along with the opiate narcotic agent, non-steroidal anti-inflammatory agent, or anesthetic. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10⁻⁹ mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

Pharmaceutical compositions containing the M2 inhibitor and/or neuraminidase inhibitor of the combination may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the cribriform plate rather than by inhalation to enable transfer of the active agents through the olfactory passages and reducing the systemic administration. Devices used for this route of administration are included in U.S. Pat. No. 6,715,485. Compositions delivered via this route may enable increased dosing or reduced total body burden reducing systemic toxicity risks associated with certain drugs.

Alternatively, the composition may be delivered by inhalation to enable transfer of the active ingredients into the respiratory system or for systemic administration through the respiratory system as exemplified in U.S. Pat. No. 6,605,302.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations.

Formulations for continuous long-term delivery are provided in, e.g., U.S. Pat. Nos. 6,797,283; 6,764,697; 6,635,268, and 6,648,083.

If desired, the M2 inhibitor may be provided in a kit. The kit can additionally include instructions for use. In some embodiments, the kit includes in one or more containers the M2 inhibitor and, separately, in one or more containers, one or more additional active agents, such as a neuraminidase inhibitor. The M2 inhibitor and additional active agents may be mixed together prior to administration or may be administered separately to the subject. Where they are administered separately to the patient they may be administered at the same time as separate formulations, at different times and over different periods of time, which may be separate from one another or overlapping. The M2 inhibitor and neuraminidase inhibitor may be administered in any order.

In other embodiments, the kit provides a means of dispensing a unit dose of the M2 inhibitor and optionally, other active agents. In one embodiment, the kit comprises an M2 inhibitor in a package configured to accommodate a pre-packaged second agent, such as a neuraminidase inhibitor, such that both agents may be dispensed concurrently for co-administration. In another embodiment, a kit comprises an M2 inhibitor in an extended release composition in the form of pellets or beads ranging from 0.5 to 5 mm in average diameter and a measuring device such as a spoon.

In other embodiments, the kit provides a combination with the M2 inhibitor and the neuraminidase inhibitor mixed in one or more containers. The kits include a therapeutically effective dose of an agent for treating flu or a flu-related condition.

Compositions of the present invention may be packaged under inert conditions such as nitrogen or noble gas to enhance shelf life. The packaging may include foil wrapping or other moisture and or light barriers to maximize shelf life of the compositions. Such measures may extend the shelf life to 2, 3, 4, 5, 6, 7, or more years. Such aggressive packaging with foil and or tough impermeable films may require special designs for ease of opening. Packaging may have two compartments, one for the M2 inhibitor, and one for an accompanying drug i.e., a neuraminidase inhibitor. It may have numbered compartments e.g., a calendar blister pack with two levels of seals for individual and daily doses so the treatments can be conveniently handled, carried around, and dispensed with reduced probability of dosing mistakes to aid compliance with the course of therapy. Alternatively the M2 inhibitor and a second agent, such as a neuraminidase inhibitor, can be placed in a single gel cap isolated from each other for increased stability by physical form, e.g. coated pellets or coated traditional or mini tablets to further increase compliance for a combination treatment.

The kit or dosage form can be structured or packaged in advance for ready distribution by mail, UPS, FEDEX or other rapid delivery in the event of a pandemic. Such packaging may, for example, comprise an outer area to accommodate a mailing label.

Preparation of a pharmaceutical composition for delivery in a subdermally implantable device can be performed using methods known in the art, such as those described in, e.g., U.S. Pat. Nos. 3,992,518; 5,660,848; and 5,756,115.

Any subject experiencing or at risk of experiencing flu or a flu-related disorder may be treated using the combinations and methods described herein.

In one embodiment of the invention, the combination of the invention may be administered to a subject who has been symptomatic for viral influenza for a specified period of time, e.g. for more than 48 hours. This may be particularly useful to treat or prevent flu-related conditions, such as a lower respiratory tract infection or otitis media.

Post-exposure treatment or prevention is also appropriate for high risk subjects who have been exposed to an individual afflicted with influenza during a time period when the individual was likely to be shedding virus and therefore, was infectious. Suitably, the combination of the invention is administered within two weeks of the subject's exposure, and preferably within four days.

Using an extended release amantadine formulation (at a constant daily dose of 250 mg, for example), a therapeutically effective steady state concentration may be achieved without using a dose escalating regimen. Such concentration is predicted to be achieved within 3 days of the onset of therapy. The slope during each absorption period for the extended release formulation is less (i.e. not as steep) as the slope for the immediate release formulation. Accordingly, the dC/dt of the extended release formulation is reduced relative to the immediate release formulation even though the dose administered is larger than for the immediate release formulation. Similarly, the extended release methods described herein may be employed to reduce the dC/dT for other M2 inhibitors or neuraminidase inhibitors enabling the administration of the combinations without the requirement for dose escalation.

The invention will be illustrated in the following non-limiting examples in which all parts and percentages are by weight unless otherwise specified.

### EXAMPLE 1

### In Vitro Method for Determining Optimal Steady-State Concentration Ratio (C_{ratio,ss})

A dose ranging study can be performed using, for example, plaque inhibition assay for drug susceptibility described by Hayden et al. (see Antimicrobial Agents and Chemotherapy (1980) 17(5):865-70). An isobolic experiment ensues in which the drugs are combined in fractions of their EDXXs to add up to ED100 (e.g., ED50:ED50 or ED25:ED75). The plot of the data is constructed. The experiment points that lie below the straight line between the ED50 points on the graph are indicative of synergy, points on the line are indicative of additive effects, and points above the line are indicative of inhibitory effects. The point of maximum deviation from the isobolic line is the optimal ratio. This is the optimal steady state ratio (Cratio,ss) and is adjusted based upon the agents half-life. Similar protocols may be applied in a wide variety of validated models.

### EXAMPLE 2

### Combinations of an M2 Inhibitor and a Neuraminidase Inhibitor

Representative combination ranges and ratios are provided below for compositions of the invention. The ranges given in Table 3 are based on the formulation strategies described herein.

TABLE 3
Adult Dosage and Ratios for Combination Therapy

| | Neuraminidase inhibitor Quantity, mg/day/ | |
|---|---|---|
| | (Neuraminidase inhibitor:M2 inhibitor Ratio Range) | |
| | | Zanamivir/ |
| M2 inhibitor mg/day | Oseltamivir/TAMIFLU ® | RELENZA ® |
| Amantadine 50-400 | 75-150/(0.38-3.0) | 10-20/(0.5-0.4) |
| Rimantadine 50-200 | 75-150/(0.19-3.0) | 10-20/(0.025-0.4) |

### EXAMPLE 3

### Release Profile of Amantadine and Oseltamivir

Release proportions are shown in Table 4 below for a combination of amantadine and oseltamivir. The cumulative fraction is the amount of drug substance released from the formulation matrix to the serum or gut environment (e.g., U.S. Pat. No. 4,839,177) or as measured with a USP II Paddle system using water as the dissolution medium.

TABLE 4
Release profile of amantadine and oseltamivir

| | Amantadine | Oseltamivir |
|---|---|---|
| | T½ = 15 hrs | T½ = 3 hrs |
| Time | cum. fraction A | Cum. Fraction B |
| 1 | 0.2 | 0.1 |
| 2 | 0.3 | 0.15 |
| 4 | 0.4 | 0.2 |
| 8 | 0.5 | 0.35 |
| 12 | 0.6 | 0.5 |
| 16 | 0.7 | 0.65 |
| 20 | 0.8 | 0.8 |
| 24 | 0.9 | 1 |

### EXAMPLE 4

### Tablet Containing a Combination of Amantadine and Oseltamivir

An extended release dosage form for administration of amantadine and oseltamivir can be prepared as three individual components. Three individual compressed tablets can be prepared, each having a different release profile, and encapsulated into a gelatin capsule which is then closed and sealed. The components of the three tablets are as follows.

TABLE 5
Tablet 1: Immediate Release (IR) Dosage Form

| Component | Function | Amount per tablet | |
|---|---|---|---|
| Amantadine | Active agent | 0 | mg |
| Oseltamivir | Active agent | 20.5 | mg |
| Dicalcium phosphate dehydrate | Diluent | 53.2 | mg |
| Microcrystalline cellulose | Diluent | 53.2 | mg |
| Sodium starch glycolate | Disintegrant | 2.4 | mg |
| Magnesium Stearate | Lubricant | 1.2 | mg |

TABLE 6
Tablet 2: Delayed Release (3-5 hours) Dosage Form

| Component | Function | Amount per tablet | |
|---|---|---|---|
| Amantadine | Active agent | 20 | mg |
| Oseltamivir | Active agent | 20.5 | mg |
| Dicalcium phosphate dehydrate | Diluent | 53.2 | mg |
| Microcrystalline cellulose | Diluent | 53.2 | mg |
| Sodium starch glycolate | Disintegrant | 2.4 | mg |
| Magnesium Stearate | Lubricant | 1.2 | mg |
| Eudragit RS30D | Delayed release | 9.52 | mg |
| Talc | Coating component | 6.6 | mg |
| Triethyl citrate | Coating component | 1.9 | mg |

TABLE 7
Tablet 3: Delayed Release (7-10 hours) Dosage Form

| Component | Function | Amount per tablet | |
|---|---|---|---|
| Amantadine | Active agent | 25.0 | mg |
| Oseltamivir | Active agent | 10.25 | mg |
| Dicalcium phosphate dehydrate | Diluent | 53.2 | mg |
| Microcrystalline cellulose | Diluent | 53.2 | mg |
| Sodium starch glycolate | Disintegrant | 2.4 | mg |
| Magnesium Stearate | Lubricant | 1.2 | mg |
| Eudragit RS30D | Delayed release | 13.0 | mg |
| Talc | Coating component | 8.8 | mg |
| Triethyl citrate | Coating component | 2.54 | mg |

The tablets are prepared by wet granulation of the individual drug particles and other core components as may be done using a fluid-bed granulator, or are prepared by direct compression of the admixture of components. Tablet 1 (Table 5) is an immediate release dosage form, releasing the active agents within 1-2 hours following administration. It contains no amantadine to avoid the dC/dT effects of the current dosage forms. Tablets 2 (Table 6) and 3 (Table 7) are coated with the delayed release coating material as may be carried out using conventional coating techniques such as spray-coating or the like. The specific components listed in the above tables may be replaced with other functionally equivalent components, e.g., diluents, binders, lubricants, fillers, coatings, and the like.

Oral administration of the capsule to a patient will result in a release profile having three pulses, with initial release of oseltamivir from the first tablet being substantially immediate, release of the amantadine and oseltamivir from the second tablet occurring 3-5 hours following administration, and release of the amantadine and oseltamivir from the third tablet occurring 7-10 hours following administration.

### EXAMPLE 5

### Pellets Containing Amantadine or Oseltamivir

Amantadine HCl (or oseltamivir) containing pellets can be prepared by wet massing. Amantadine HCl (or oseltamivir) can be weighed and sieved through a No. 20 screen into the bowl of low shear planetary mixer. To this, microcrystalline cellulose can be weighed and added through No. 20 screen and blended with Amantadine HCl (or oseltamivir) using a spatula, then in a planetary mixer on low speed. Eudragit NE 400, accurately weighed can be incrementally added to the powder blend, allowing sufficient time between additions for complete distribution. To avoid accumulation at the bottom and to loosen the material, the bottom can be periodically scraped. Purified water can be blended into the mixture in 10 mL increments (the first of which can be used to rinse the beaker containing Eudragit NE 40D) until a uniform blend appropriate for extrusion was obtained. Experimental batches can be prepared with 10 to 50 ml water. Wet massing is followed by extrusion, spheronization and drying by procedures well known in the prior art.

TABLE 8
Pellets containing Amantadine HCl

| | | | Wt. solid per | Target Wt. | Actual Wt. |
|---|---|---|---|---|---|
| | | Percent in | Batch | per Batch | per Batch |
| Component | Supplier | Formula¹ | (grams) | (g) | (g) |
| Amantadine HCl | | 20.0% | 50.0 | 50.0 | 50.00 |
| Eudragit NE 40D | Degussa | 30.0% | 75.0 | 187.5 | 187.50 |
| Microcrystalline | FMC Corp | 50.0% | 125.0 | 125.0 | 125.00 |
| Cellulose (Avicel PH | | | | | |
| 101) | | | | | |
| Purified Water | | N/A | N/A | 50.0 | 10.0 |
| | TOTAL | 100.0% | 250.0 | N/A | N/A |

¹based on solid in the final product

TABLE 9
Pellets Containing Oseltamivir

| | | | Wt. solid per | Target Wt. | Actual Wt. |
|---|---|---|---|---|---|
| | | Percent in | Batch | per Batch | per Batch |
| Component | Supplier | Formula¹ | (grams) | (g) | (g) |
| Oseltamivir | | 20.0% | 40.0 | 40.0 | 39.98 |
| Eudragit NE 40D | Degussa | 30.0% | 60.0 | 150.0 | 150.05 |
| Microcrystalline Cellulose | FMC Corp | 50.0% | 100.0 | 100.0 | 100.00 |
| (Avicel PH 101) | | | | | |
| Purified Water | | N/A | N/A | 50.0 | 10.0 |
| | TOTAL | 100.0% | 200.0 | N/A | N/A |

¹based on solid in the final product

### EXAMPLE 6

### Amantadine HCl/Oseltamivir Formulations

Formulations of Sustained Release (SR) Amantadine HCl (or Oseltamivir), fast and medium, can be obtained by applying a subcoat of Opadry (2% final pellet weight) followed by a functional coating of Surelease (15% dispersion prepared from 25% Surelease) to 20% Amantadine HCl (or Oseltamivir) pellets.

Formulations of Sustained Release (SR) Amantadine HCl (or Oseltamivir), slow, can be obtained by applying a subcoat of Opadry (10% final bead weight), functional coating of plasticized Eudragit RS (35% final pellet weight) and triethylcitrate (plasticizer, 10% of the functional coating) to 20% Amantadine HCl (or Oseltamivir) pellets.

TABLE 10
Amantadine SR Products

| | | | SR Amantadine | | |
|---|---|---|---|---|---|
| | SR Amantadine | | Pellets | SR Amantadine | |
| Product | Pellets (Fast) | | (Medium) | Pellets (Slow) | |
| “Label Claim” (mg | 0.164 | | Blend of 40% | 0.100 | |
| active/mg pellets) | | | “Fast” and | | |
| Sample weight (mg | 134.6 | 136.2 | 60% “Slow” | 207.9 | 208.9 |
| pellets) | | | | | |
| 16 hr “Assay” Value | 23.41 | 23.44 | | 17.97 | 18.24 |
| (mg released) | | | | | |
| “Assay” Value (mg | 0.174 | 0.172 | | 0.0864 | 0.0873 |
| active/mg pellets) | | | | | |

| | | | | |
|---|---|---|---|---|
| Average Assay Value | 0.173 | | | 0.0869 |
| (mg active/mg | | | | |
| pellets) | | | | |
| Amount of pellets for | 520.0 | 208.0 | 621.6 | 1036 |
| 90 mg dose (mg) | | | | |

TABLE 11
Oseltamivir Immediate Release (IR) Product

| Product | IR Oseltamivir |
|---|---|
| “Label Claim” (mg active/mg granulation) | 0.40 |
| Amount of granulation for 75 mg dose (mg) | 187.5 |

TABLE 12
Oseltamivir SR Product

| | SR Oseltamivir | SR Oseltamivir | SR Oseltamivir |
|---|---|---|---|
| Product | Pellets (Fast) | Pellets (Medium) | Pellets (Slow) |
| “Label Claim” | 0.36 | 0.32 | 0.30 |
| (mg active/mg | | | |
| pellets) | | | |
| Amount of pellets | 208.3 | 234.4 | 250.0 |
| for 75 mg dose (mg) | | | |

### EXAMPLE 7

### Dosage Formulation of Amantadine-Oseltamivir Combination

Various combinations of amantadine and oseltamivir can be prepared by filling the respective pellets in hard gelatin capsules as shown in Table 13. The separately prepared pellets provide the flexibility of making ratios of amantadine to oseltamivir pellets ranging from 1:1000 to 1000:1, more preferably from 1:100 to 100:1, even more preferably 1:10 to 10:1, most preferably 1:4 to 4:1. Note that the masses for capsules prepared according to Table 13 would be divided into multiple capsules to facilitate administration of the daily target dose of each drug, so that when taken together, once per day the targeted amount of 90 mg amantadine and 75 mg oseltamivir per day is administered.

TABLE 13
Amantadine-Oseltamivir Dosage Combinations

| | Amantadine | | Oseltamivir | |
|---|---|---|---|---|
| | Wt. | | Wt. | |
| | solid/dosage | | solid/dosage | |
| Product | Unit (mg) | Formulation | Unit (mg) | Formulation |
| NPI-89011a | 520.0 | SR (Fast) | 187.5 | IR |
| NPI-8901b | 208 | SR (Fast) | 187.5 | IR |
| | 621.6 | SR (Slow) | | |
| NPI-8901c | 1036 | SR (Slow) | 208.3 | IR |
| NPI-8901d | 520 | SR (Fast) | 208.3 | SR (Fast) |
| NPI-8901e | 208 | SR (Fast) | 208.3 | SR (Fast) |
| | 621.6 | SR (Slow) | | |
| NPI-8901f | 1036 | SR (Slow) | 208.3 | SR (Fast) |
| NPI-8901g | 520 | SR (Fast) | 234.4 | SR (Medium) |
| NPI-8901h | 208 | SR (Fast) | 234.4 | SR (Medium) |
| | 621.6 | SR (Slow) | | |
| NPI-8901i | 1036 | SR (Slow) | 234.4 | SR (Medium) |
| NPI-8901j | 520 | SR (Fast) | 250 | SR (Slow) |
| NPI-8901k | 208 | SR (Fast) | 250 | SR (Slow) |
| | 621.6 | SR (Slow) | | |
| NPI-8901l | 1036 | SR (Slow) | 250 | SR (Slow) |

SRS = Sustained Release,
IR = Immediate Release

### EXAMPLE 8

### Dissolution Profiles

The dissolution profiles of the various amantadine-oseltamivir combinations (as shown in Example 7) can be obtained from USP II (paddle) dissolution system at 50 rpm, at a temperature of 37.0±0.5° C., using water as the medium.

For the dissolution analysis, 10 mL dissolution solutions of amantadine are diluted with 3 mL of 0.1% formic acid. Standards of amantadine are also prepared and diluted with 3 mL of 0.1% formic acid. A 1 mL aliquot of the diluted solution or standard is then transferred into an HPLC vial. A 10 μL aliquot of the solution or standard is then injected onto the LC/MS/MS for analysis. A C18 reversed phase column (Phenomenex, Luna 5μ, Phenyl-Hexyl 50×2 mm) can be used for analysis. Amantadine can be separated from endogenous interfering substances and subsequently eluted from the HPLC column by a mobile phase of 33% acetonitrile, 33% methanol and 34% formic acid for mass quantification. A mass spectrometer can be used to detect and quantify amantadine. Data can be processed and calculated by an automated data acquisition system (e.g., Analyst 1.2, Applied Biosystems, Foster City, Calif.).

### EXAMPLE 9

### Release Profiles of IR and SR Amantadine-Oseltamivir Formulations

In vivo release profiles can be obtained using the Gastro-Plus software package v.4.0.2. The release profiles and pharmacokinetic properties for an extended release combination product made according to Examples 5-7 as compared to IR administration of presently marketed products can be estimated using the Gastro-Plus software package. Comparisons to the manufacturers' recommendation for the IR formulations presently approved for the market will show a more rapid time to steady state concentration, particularly for amantadine. The SR product dC/dT is considerably lower than the IR form for a similar dose for both amantadine and oseltamivir. Thus, the SR formulations provide a more gradual increase in the drug during each patient dose.

In addition to achieving the desired release profile, this combination formulation will exhibit a preferred decrease Cmax/Cmean, even with a higher dose of the M2 inhibitor and neuraminidase inhibitor, thus the present invention may provide greater doses for increased therapeutic effect without escalation that might otherwise be required. Furthermore, the increased dosing allows less frequent administration of the therapeutic agents.

### EXAMPLE 10

### Multiple Dose Safety Study in Flu Patients with an Extended Release Amantadine, Extended Release Oseltamivir Combination

A study to determine safety and pharmacokinetics of an extended release combination formulation of amantadine and oseltamivir is described in Table 14.

TABLE 14

| | |
|---|---|
| Purpose | To determine the safety and pharmacokinetics of repeated doses of |
| | drug. |
| Dosage: | Based on previous single ascending dose (SAD) study, either e.g. 100 mg |
| | amantadine SR + 75 mg oseltamivir SR, 200 mg amantadine SR + 75 mg |
| | oseltamivir SR, or 200 mg amantadine SR + 150 mg |
| | oseltamivir SR, QD for 7 days |
| Concurrent | Amantadine IR or amantadine IR plus oseltamivir IR (both dosed as |
| Controls | per manufacturers' labels) |
| Route: | Oral |
| Subject | Males or females diagnosed with influenza |
| Population: | |
| Structure: | 4 arm |
| Study Sites: | TBD |
| Blinding: | Patients blinded |
| Method of | Random with equal number of males and females in each group and |
| Subject | equal age distributions within groups |
| Assignment: | |
| Total Sample | 24 Subjects 6 per dosing arm |
| Size: | |
| Primary | None |
| Efficacy | |
| Endpoint: | |
| Adverse | Monitored at least twice daily for behavioral, cardiovascular, and |
| Events: | gastrointestinal effects reported for high doses of amantadine or |
| | oseltamivir (including dizziness, headache, confusion, constipation, |
| | hypertension, coughing, nausea, diarrhea, vomiting). |
| Blood | By canula through first day of study period then 2-4 times daily for |
| Collection | rest of study |
| Analysis | Assays to measure amantadine, oseltamivir, and potentially other |
| | physiological parameters, adverse events |

### EXAMPLE 11

### Determination of Increased-Dose Tolerability for Amantadine SR Formulations

A study to determine safety and tolerability of increased dosing for Amantadine SR is described in Table 15. The study results establish a maximum administrable dose greater than 200 mg when given once per day, as well as confirm tolerability of a non-dose escalating dosing regimen (i.e., administration of substantially identical doses of amantadine throughout the term of dosing). This study may also be performed using a similarly prepared rimantadine SR formulation.

TABLE 15

| | |
|---|---|
| Purpose | Multiple Dose Tolerability |
| Dosage: | 100, 200, 300, 400, 500, 600, 700, and 800 mg amantadine SR |
| Concurrent | Placebo |
| Control: | |
| Route: | Oral |
| Subject | Healthy, drug-naive male subjects |
| Population: | |
| Structure: | Placebo-extended, Sequential dose escalation |
| Study Sites: | Single center |
| Blinding: | Open label |
| Method of | Subjects in each Cohort will be randomized to either active drug |
| Subject | (n = 8) or placebo (n = 2) |
| Assignment: | |
| Total Sample | 80-100 subjects |
| Size: | |
| Primary Efficacy | None |
| Endpoint: | |
| Adverse Events: | Monitored with reports by clinic personnel at least 2 or 3 times per |
| | day throughout the study, as well as volunteered by subjects. |
| Blood Collection | Blood sampling and plasma preparations at the following time points: |
| | Day 1: 0, 1, 2, 3, 4, 6, 7, 8, 10, 12, 14, 16, 20 |
| | Days 2-6: pre-dose trough |
| | Day 7: 0, 1, 2, 3, 4, 6, 7, 8, 10, 12, 14, 16, 20, 24, 48, 72, 96, 120, |
| | 144, and 168 hours |
| Analysis | Adverse events (including dizziness, headache, confusion, |
| | constipation, hypertension, coughing), tolerability, |
| | Pharmacokinetics |

### EXAMPLE 12

### Treating Influenza

A patient diagnosed with or suspected of having influenza A is administered 100 mg or more, up to a maximum tolerated dose (as determined using the protocol in Example 11) of amantadine or rimantadine formulated as described in Example 5, once daily for 5, 6, 7, 8, 9, 10, 12, or 14 days. A therapeutically effective steady state serum concentration is reached within three days of the start of this therapy.

### EXAMPLE 13

### Administration for Influenza Prophylaxis

A patient at risk of being infected with influenza A is administered 100 mg or more, up to a maximum tolerated dose (as determined using the protocol in Example 11) of amantadine or rimantadine formulated as described in Example 5, once daily for 12, 14, 16, 18, 20, 21, 22, 24, 26, 28, or 30 days. A prophylactically effective steady state serum concentration is reached within three days of the start of this therapy.

What is claimed is:

1. A pharmaceutical composition comprising:
(a) an M2 inhibitor;
(b) a neuraminidase inhibitor,
wherein at least one of said M2 inhibitor or said neuraminidase inhibitor is provided in an extended release dosage form and wherein at least one of said active pharmaceutical ingredients in said extended release dosage form has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium.

2. The composition of claim 1, wherein said M2 inhibitor is provided in the extended release dosage form.

3. The pharmaceutical composition of claim 1, wherein said M2 inhibitor has a dC/dT less than about 80% of the rate for the IR formulation.

4. The pharmaceutical composition of claim 1, wherein said M2 inhibitor has a Cₘₐₓ/Cₘₑₐₙ of approximately 1.6 or less approximately 2 hours to at least 12 hours after said composition is introduced into a subject.

5. The pharmaceutical composition of claim 1, wherein the relative Cratio.var of said M2 inhibitor and said neuraminidase inhibitor is less than 100% from 2 hour to 12 hours after said composition is introduced into a subject.

6. The pharmaceutical composition of claim 1, wherein the M2 inhibitor is selected from amantadine and rimantadine.

7. The pharmaceutical composition of claim 1, wherein said neuraminidase inhibitor is oseltamivir.

8. The pharmaceutical composition of claim 1, wherein said pharmaceutical composition is formulated for oral delivery.

9. The pharmaceutical composition of claim 1, wherein the M2 inhibitor is amantadine and the neuraminidase inhibitor is oseltamivir.

10. The pharmaceutical composition of claim 1, wherein the M2 inhibitor is amantadine and wherein the composition is in a unit dosage containing 100-500 mg amantadine.

11. A method of preventing or treating flu or a flu-related condition comprising administering to a subject in need thereof a therapeutically effective amount of:
(a) an M2 inhibitor; and
(b) a neuraminidase inhibitor,
wherein at least one of said M2 inhibitor or said neuraminidase inhibitor is provided in an extended release dosage form and wherein at least one of said active pharmaceutical ingredients in said extended release dosage form has an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium.

12. The method of claim 11, wherein said M2 inhibitor is provided in the extended release dosage form.

13. The method of claim 11, wherein said M2 inhibitor has a dC/dT less than about 80% of the rate for the IR formulation.

14. The method of claim 11, wherein said M2 inhibitor has a Cₘₐₓ/Cₘₑₐₙ of approximately 1.6 or less approximately 2 hours to at least 12 hours after said composition is introduced into a subject.

15. The method of claim 11, wherein said M2 inhibitor and said neuraminidase inhibitor have a relative Cratio.var of less than 100% from 2 hour to 12 hours after the M2 inhibitor and neuraminidase inhibitor are introduced into a subject.

16. The method of claim 11, wherein the relative Cratio.var of said M2 inhibitor and said neuraminidase inhibitor is less than 70% of the corresponding IR formulation from 2 hour to 12 hours after said composition is introduced into the subject.

17. The method of claim 11, wherein the M2 inhibitor is amantadine and the neuraminidase inhibitor is oseltamivir.

18. A kit comprising the pharmaceutical composition of claim 1 and instructions for administering the composition to treat or preventing flu.

19. The kit of claim 18, wherein said M2 inhibitor and said second agent are formulated as a single formulation.

20. A pharmaceutical composition comprising:
(a) amantadine in a dose ranging between 200 and 500 mg; and
(b) oseltamivir in a dose ranging between 75 and 150 mg;
wherein amantadine and oseltamivir are both provided in an extended release dosage form, said extended release dosage form having an in vitro dissolution profile less than 70% in one hour, less than 90% in two hours, greater than 40% in six hours, and greater than 85% in 12 hours as measured using a USP type 2 (paddle) dissolution system at 50 rpm, at a temperature of 37±0.5° with water as a dissolution medium;
